(19) United States
(12) Patent Application Publication
(10) Pub. No.: US 2023/0301627 A1
(43) Pub. Date: Sep. 28, 2023

(54) NEEDLE GUIDANCE DEVICE FOR ULTRASOUND PROBE

(71) Applicants: Eastern Virginia Medical School, Norfolk, VA (US); Old Dominion University, Norfolk, VA (US)

(72) Inventors: Michael Morgan Guju, Norfolk, VA (US); Sebastian Y Bawab, Norfolk, VA (US); Michael Polanco, Norfolk, VA (US); Christopher Michael O'Brien, Norfolk, VA (US); Payton Blodgett, Norfolk, VA (US)

(21) Appl. No.: 18/127,462

(22) Filed: Mar. 28, 2023

Related U.S. Application Data
(60) Provisional application No. 63/324,378, filed on Mar. 28, 2022.

(51) Int. Cl. A61B 8/00

(52) U.S. Cl. CPC A61B 8/4455; A61B 2017/3413

(57) ABSTRACT

Various implementations include an ultrasound needle guidance device. The device includes a coupling body and at least one needle holder portion. The coupling body is configured to fixedly couple to an ultrasound probe that is able to produce an ultrasonic beam along a scanning plane. The needle holder portion has a rotational axis and defines a needle opening having an opening central axis extending perpendicularly to the rotational axis. The needle holder portion is rotationally coupled to the coupling body such that the needle holder portion is rotatable about the rotational axis relative to the coupling body from a first position to a second position. The needle holder portion is configured such that, when the coupling body is coupled to the ultrasound probe, the opening central axis is disposed within the scanning plane in all positions in a range from the first position to the second position.

### CROSS-REFERENCE TO RELATED APPLICATION

This application claims the benefit of priority to U.S. Provisional Application No. 63/324,378 filed on Mar. 28, 2022, which is herein incorporated by reference in its entirety for all purposes.

### BACKGROUND

Point of care ultrasound is an imaging technique that is being more frequently utilized to assist in diagnosis and treatment of disease. One of the major uses in the clinic and the hospital is its use in guiding injections. Once the anatomic structure is identified on US, the needle is inserted and advanced. Metal needles are bright (hyperechoic) on ultrasound, making them easily visible when in the field of view. When done properly, this characteristic paired with the real time nature of ultrasound allows physicians to place needles and deliver medications to exact areas with great confidence.

A significant challenge when performing an ultrasound guided injection is continuous visualization of the needle during the entire procedure. The ultrasound beam can penetrate several inches deep and is a few inches wide, but it is approximately 1 mm thick. If the needle moves out of the field of view either by advancing the needle or by the ultrasound probe deviating from its position, it can be dangerous to advance the needle. Advancing the needle out of the field of view can result in complications such as failed nerve blocks, pneumothorax, or intravascular injection.

When the physician sees the needle and advances it with one hand and holds the ultrasound probe in the other hand, this is called the “freehand technique.” This is currently the most common method utilized in practice. But there can be ways to improve these ultrasound guided procedures.

There are currently ultrasound needle guidance systems that are standard of care for certain procedures, such as prostate biopsies. The concept of these guidance systems is to couple the movement of the needle to the probe to keep the needle in the field of view and to assist in the trajectory of the needle. Current needle guidance devices have fixed angles for which to advance the needle (e.g., the device is pre-set to 45 degrees).

Thus, there is a need for a needle guidance device for ultrasound that allows for a variable selection of needle insertion angles.

### BRIEF SUMMARY

According to some aspects, an ultrasound needle guidance device comprises a coupling body configured to fixedly couple to an ultrasound probe, wherein the ultrasound probe is able to produce an ultrasonic beam along a scanning plane; and at least one needle holder portion, wherein the needle holder portion has a rotational axis and defines a needle opening having an opening central axis extending perpendicularly to the rotational axis, wherein the needle holder portion is rotationally coupled to the coupling body such that the needle holder portion is rotatable about the rotational axis relative to the coupling body from a first position to a second position, wherein the needle holder portion is configured such that, when the coupling body is coupled to the ultrasound probe, the opening central axis is disposed within the scanning plane in all positions in a range from the first position to the second position.

In some aspects, in addition to the preceding aspect, the needle holder portion comprises a round cross-sectional shape in a plane perpendicular to the rotational axis.

In some aspects, in addition or in the alternative to the preceding aspects, the needle holder portion comprises one or more protrusions extending radially relative to the rotational axis, wherein the needle opening extends through the protrusion.

In some aspects, in addition or in the alternative to the preceding aspects, the ultrasound probe has a scanning surface plane through which the ultrasonic beam passes, wherein the scanning plane is perpendicular to the scanning surface plane, wherein the opening central axis is disposed at a 0-degree angle relative to the scanning surface plane in the first position.

In some aspects, in addition or in the alternative to the preceding aspects, the opening central axis is disposed at a 90-degree angle relative to the scanning surface plane in the second position.

In some aspects, in addition or in the alternative to the preceding aspects, the needle opening has a diameter of 0.203 mm or greater. For example, in some aspects, the needle opening has a diameter of 0.280 mm or greater, a diameter of 0.642 mm or greater, a diameter of 0.718 mm or greater, a diameter of 1.270 mm or greater, or a diameter of 1.473 mm or greater.

In some aspects, in addition or in the alternative to the preceding aspects, the needle opening is a first needle opening having a first opening central axis, and the needle holder portion further defines a second needle opening having a second opening central axis extending perpendicularly to the rotational axis, wherein the needle holder portion is configured such that, when the coupling body is coupled to the ultrasound probe, the second opening central axis is disposed within the scanning plane in all positions in the range from the first position to the second position.

In some aspects, in addition or in the alternative to the preceding aspects, the first needle opening has a first diameter and the second needle opening has a second diameter that is different than the first diameter.

In some aspects, in addition or in the alternative to the preceding aspects, the needle holder portion is removably couplable to the coupling body.

In some aspects, in addition or in the alternative to the preceding aspects, the needle holder portion comprises a first portion and a second portion that is separable from the first portion, wherein at least one surface of the first portion and at least one surface of the second portion abut each other when the needle holder portion is coupled to the coupling body, wherein the at least one surface of the first portion and the at least one surface of the second portion define the needle opening.

In some aspects, in addition or in the alternative to the preceding aspects, the coupling body includes an inner surface configured to face the ultrasound probe when the device is coupled to the ultrasound probe, wherein the inner surface of the coupling body includes a protrusion configured to engage at least one feature of the ultrasound probe.

In some aspects, in addition or in the alternative to the preceding aspects, the coupling body includes an inner surface configured to face the ultrasound probe when the device is coupled to the ultrasound probe, wherein the inner surface of the coupling body defines an opening configured to engage at least one feature of the ultrasound probe.

In some aspects, in addition or in the alternative to the preceding aspects, the coupling body further includes a latch configured to engage at least one feature of the ultrasound probe. For example, in some aspects, the latch is resiliently biased toward an engagement position in which the latch is configured to engage the at least one feature of the ultrasound probe and is urgable toward a disengagement position in which the latch is configured to disengage the at least one feature of the ultrasound probe.

In some aspects, in addition or in the alternative to the preceding aspects, the coupling body comprises a resilient material.

### BRIEF DESCRIPTION OF THE DRAWINGS

Example features and implementations are disclosed in the accompanying drawings. However, the present disclosure is not limited to the precise arrangements and instrumentalities shown.

FIG. 1 shows a side view of an ultrasound needle guidance device, according to one implementation, coupled to an ultrasound probe.

FIG. 2 shows a perspective view of the device shown in FIG. 1 decoupled from the ultrasound probe.

FIG. 3 shows a side view of the device of FIG. 1.

FIG. 4 shows a top view of the device of FIG. 1.

FIG. 5 shows a side view of an ultrasound needle guidance device, according to another implementation, coupled to an ultrasound probe.

FIG. 6 shows a perspective view of the device of FIG. 5.

FIG. 7 shows a perspective view of the needle holder portion of an ultrasound needle guidance device, according to another implementation.

FIG. 8 shows a perspective view of the needle holder portion of an ultrasound needle guidance device, according to another implementation.

FIG. 9 shows a perspective view of the needle holder portion of an ultrasound needle guidance device, according to another implementation.

FIG. 10 shows a perspective view of an ultrasound needle guidance device, according to another implementation, decoupled from the ultrasound probe.

### DETAILED DESCRIPTION

The devices, systems, and methods disclosed herein provide for an ultrasound needle guidance device that attaches to an ultrasound probe and includes a “compass wheel” design which allows the proceduralist to use feel and real-time ultrasound imaging to choose any angle they see most appropriate. A needle disposed within the needle holder of the device can be angled relative to a scanning surface of the probe while maintaining the needle within the scanning plane of the probe. The benefits of this design are that it aids the injector to keep the needle tip in field of view, allows sufficient needle angle variability, assists in needle trajectory, decreases time needed to reach targets, increases user confidence and comfort with procedures, and increases accuracy of needle placement.

Various implementations include an ultrasound needle guidance device. The device includes a coupling body and at least one needle holder portion. The coupling body is configured to fixedly couple to an ultrasound probe that is able to produce an ultrasonic beam along a scanning plane. The needle holder portion has a rotational axis and defines a needle opening having an opening central axis extending perpendicularly to the rotational axis. The needle holder portion is rotationally coupled to the coupling body such that the needle holder portion is rotatable about the rotational axis relative to the coupling body from a first position to a second position. The needle holder portion is configured such that, when the coupling body is coupled to the ultrasound probe, the opening central axis is disposed within the scanning plane in all positions in a range from the first position to the second position.

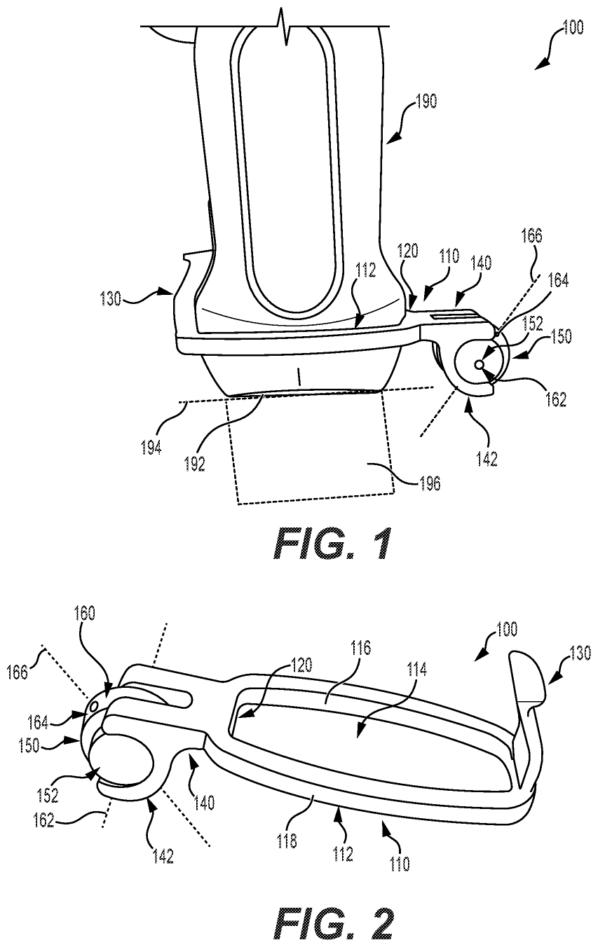

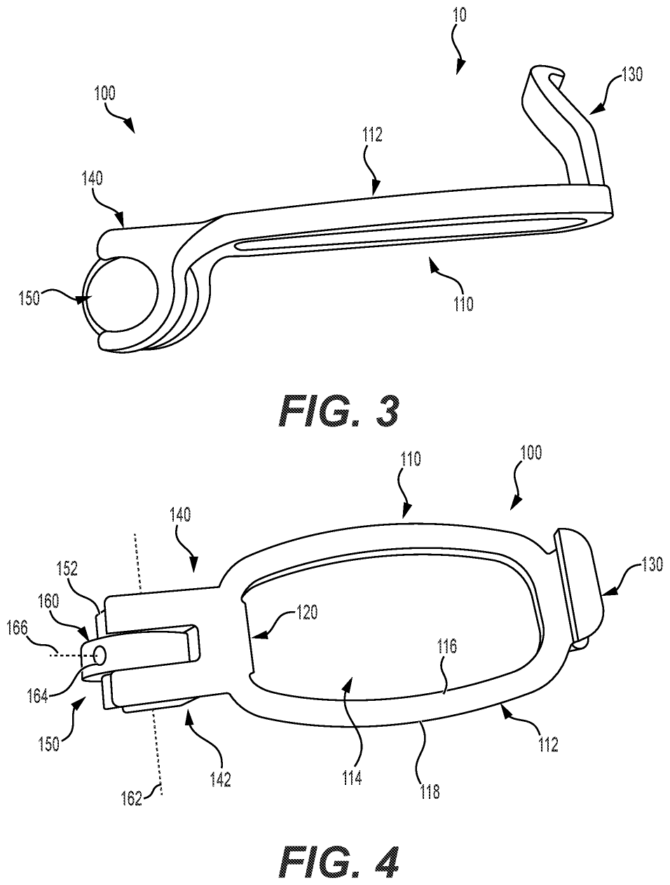

FIGS. 1-4 show an ultrasound needle guidance device 100, according to various aspects of some implementations. The device 100 includes a coupling body 110 and a needle holder portion 150.

The device 100 is shown fixedly coupled to an ultrasound probe 190. As used herein, the term “fixedly” means “non-rotatable” relative to another feature or structure. The ultrasound probe 190 has a scanning surface 192 defining a scanning surface plane 194 through which the ultrasonic beam passes. The ultrasound probe 190 produces an ultrasonic beam along a scanning plane 196 that is perpendicular to the scanning surface plane 194.

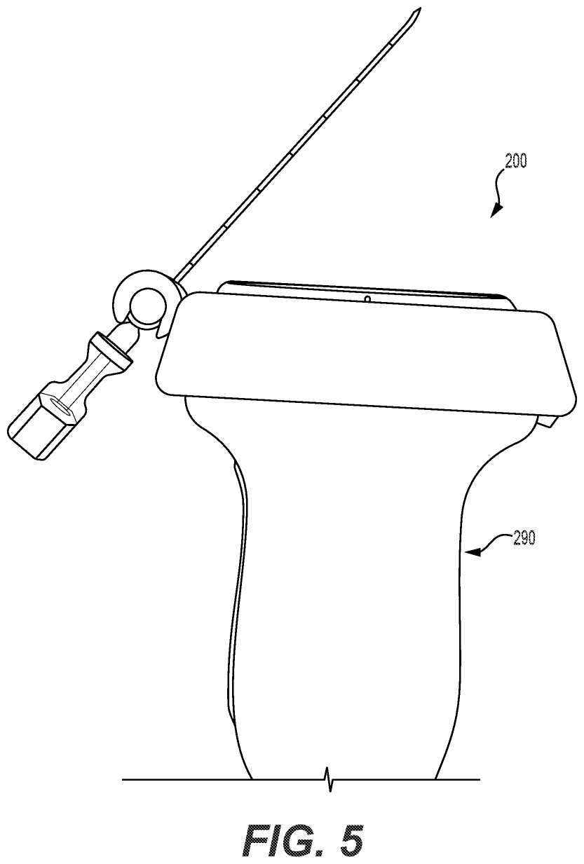

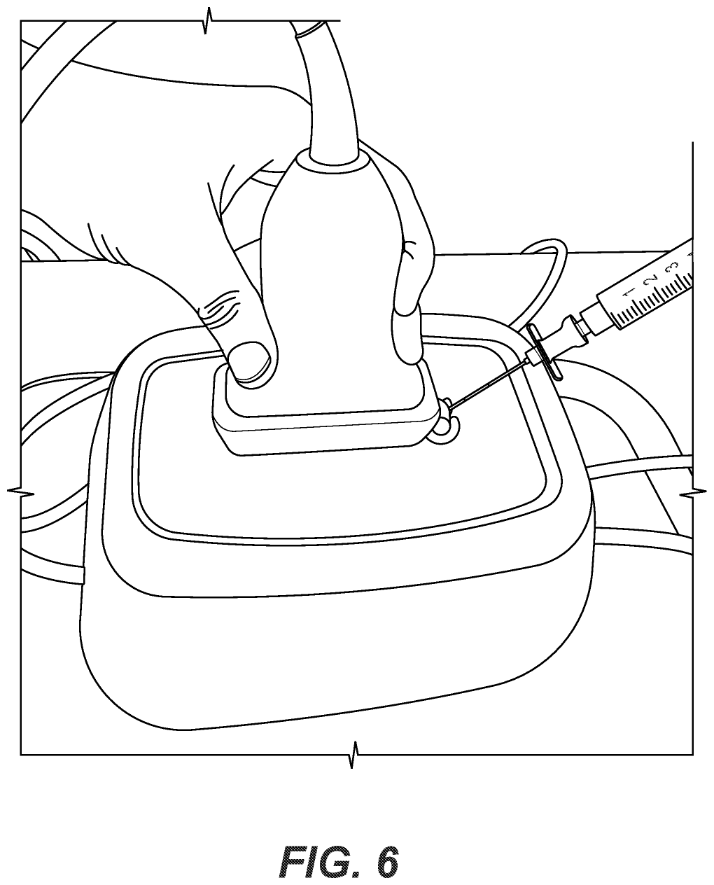

The coupling device 110 includes a collar 112, a protrusion 120, a latch 130, and a holder support 140. The collar 112 defines a probe opening 114 that has an inner surface 116 configured to face the ultrasound probe 190 when the device 100 is coupled to the ultrasound probe 190 and an outer surface 118 opposite the inner surface 116. The probe opening 114 is shaped to fit around at least a portion of a specific ultrasound probe 190 and can be easily modified to be coupled to any shape ultrasound probe 190. For example, FIGS. 5 and 6 show another implementation of an ultrasound needle guidance device 200 configured to be couplable to a different ultrasound probe 290.

The protrusion 120 shown in FIGS. 1-4 extends inwardly from the inner surface 116 of the collar 112 of the coupling body 110. The protrusion 120 is configured to engage a groove of the surface of the ultrasound probe 190. However, for implementations of devices designed to be used with different ultrasound probes, the protrusion can be configured to engage any other feature of the ultrasound probe. In some implementations, the protrusion is configured to engage two or more features of the ultrasound probe. In some implementations, the inner surface of the coupling body includes two or more protrusions configured to engage one or more features of the ultrasound probe.

In some implementations, the inner surface of the coupling body defines an opening configured to engage a protrusion of the ultrasound probe. In some implementations, the opening can be configured to engage any other feature of the ultrasound probe. In some implementations, the opening is configured to engage two or more features of the ultrasound probe. In some implementations, the inner surface of the coupling body defines two or more openings configured to engage one or more features of the ultrasound probe.

The latch 130 is configured to engage at least one feature of the ultrasound probe 190 and is designed to allow the device 100 to be quickly coupled to and decoupled from the ultrasound probe 190. The latch 130 is resiliently biased toward an engagement position in which the latch 190 is configured to engage the feature of the ultrasound probe 190 and is urgable toward a disengagement position in which the latch 130 is configured to disengage from the feature of the ultrasound probe 190. The latch 130 extends from a portion of the collar 112 opposite the protrusion 120 such that the protrusion 120 and latch 130 can engage opposite sides of the ultrasound probe 190 to securely couple the coupling body 110 to the probe 190.

Although the latch 130 shown in FIGS. 1-4 is configured to engage a protrusion of the ultrasound probe 190, for implementations of devices designed to be used with different ultrasound probes, the latch can be configured to engage any other feature of the ultrasound probe. In some implementations, the latch is configured to engage two or more features of the ultrasound probe. In some implementations, the coupling body includes two or more latches configured to engage one or more features of the ultrasound probe.

The holder support 140 includes two arms 142 extending from the outer surface 118 of the collar 110. The arms 142 are arcuate and have an inner radius of curvature substantially the same as the radius of curvature of portions 152 of the needle holder portion 150 such that the portions 152 can be disposed within the arms 142 of holder support 140 to couple the needle holder portion 150 to the coupling body 110. The arms 142 of the holder support 140 are made of a resilient material to allow the arms 142 to be partially straightened to increase the radius of curvature of the arms 142 to release the portions 152 of the needle holder portion 150.

The collar 112, protrusion 120, latch 130, and holder support 140 of the coupling body 110 shown in FIGS. 1-4 are made of a resilient material, such as a polymer. However, in other implementations, one or more of the collar, protrusion, and latch of the coupling body can be made of any other material.

The needle holder portion 150 has a cylindrical body 160 that has a rotational axis 162 extending along the central longitudinal direction of the cylindrical body 160. The body 160 defines a needle opening 164 having an opening central axis 166 extending perpendicularly to, and intersecting, the rotational axis 162. The needle opening 164 shown in FIGS. 1-4 has a diameter of 1.270 mm such that an 18-gauge needle will fit into the opening 164. However, in other implementations, the needle opening can have any sized diameter to fit any desired gauge needle, such as a diameter of 0.203 mm or greater for a 27-gauge needle, a diameter of 0.280 mm or greater for a 25-gauge needle, a diameter of 0.642 mm or greater for a 23-gauge needle, a diameter of 0.718 mm or greater for a 22-gauge needle, or a diameter of 1.473 mm or greater for a 17-gauge needle.

As discussed above, portions 152 of the body 160 of the needle holder portion 150 have a radius of curvature about the rotational axis 162 that is substantially the same as the radius of curvature of the inner surface of the arms 142 of the holder support 140. The needle holder portion 150 is removably couplable to the coupling body 110 by disposing the portion 152 of the body 150 within the arms 142 of the holder support 140. If a different diameter needle opening is desired, the needle holder portion 150 can be removed from the coupling body 110 of the device 100 and a different needle holder portion defining a different diameter needle opening can be installed.

When the body 160 is disposed within the arms 142 of the holder support 140, the needle holder portion 150 is rotationally coupled to the coupling body 110 such that the needle holder portion 150 is rotatable about the rotational axis 162 relative to the coupling body 110 from a first position to a second position. The needle holder portion 150 is configured such that, when the coupling body 110 is coupled to the ultrasound probe 190, the opening central axis 166 is disposed within the scanning plane 196 in all positions in a range from the first position to the second position. For the device 100 shown in FIGS. 1-4, the opening central axis 166 is disposed at a 0-degree angle relative to the scanning surface plane 194 in the first position, and the opening central axis 166 is disposed at a 90-degree angle relative to the scanning surface plane 194 in the second position. The body 160 of the needle holder portion 150 is selectably rotatable to any position between the first and the second positions depending on a desired needle entry angle.

Although the body 160 of the needle holder portion 150 shown in FIGS. 1-4 includes a round cross-sectional shape in a plane perpendicular to the rotational axis 162, in other implementations, the body of the needle holder portion can have any cross-sectional shape.

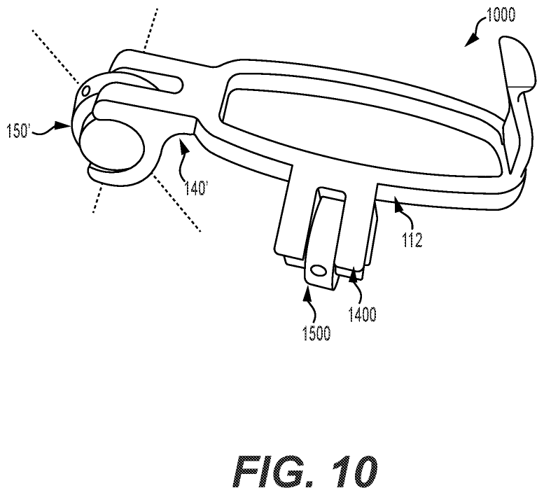

Although the device 100 shown in FIGS. 1-4 shows one holder support 140 and one needle holder portion 150, devices according to other implementations may include more than one holder support and needle holder portion. For example, in the implementation shown in FIG. 10, the device 1000 is similar to device 100 shown in FIGS. 1-4 but includes a second holder support 1400 and a second needle holder portion 1500 that are spaced apart along the circumference of the collar 112 from the other holder support 140′ and needle holder portion 150′.

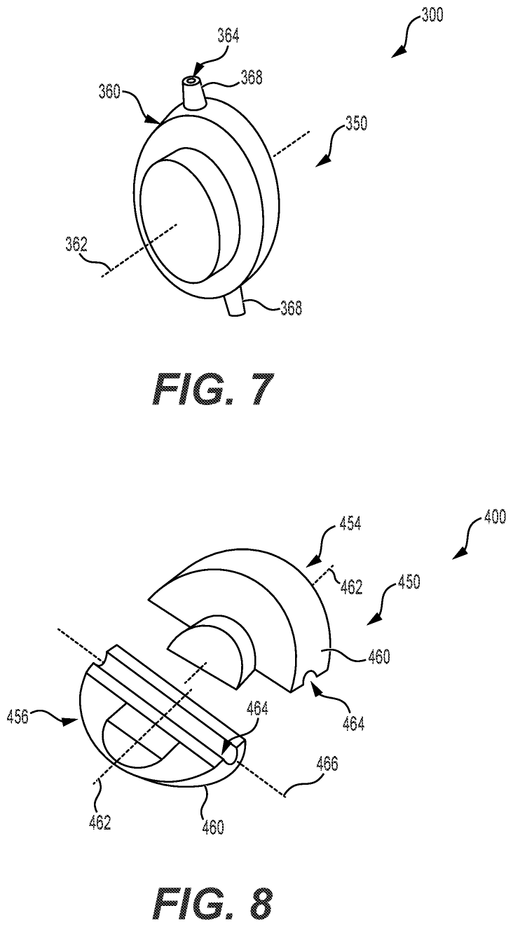

In implementations, such as the device 300 shown in FIG. 7, the body 360 of the needle holder portion 350 can include one or more protrusions 368 extending radially relative to the rotational axis 362. The needle opening 364 extends through the protrusion 368 to provide additional support to a needle disposed within the needle opening 364. The protrusion 368 effectively provides for a larger diameter of the body 360 of the needle holder portion 350 in the area around the needle opening 364.

FIG. 8 shows another implementation of a device 400 including a two-piece needle holder portion 450. The needle holder portion 450 includes a first portion 454 and a second portion 456 that is separable from the first portion 454. A surface of the first portion 454 and a surface of the second portion 456 abut each other when the needle holder portion 450 is coupled to the coupling body 410. The first portion 454 and the second portion 456 are separated along a plane that extends along the rotational axis 462 of the body 460 and the opening central axis 466 of the needle opening 464. Thus, each of the first portion 454 and the second portion 456 combine to define the needle opening 464.

Although the first portion 454 and the second portion 456 are separated along a plane that extends along the rotational axis 462 and the opening central axis 466, in other implementations, the first portion and the second portion are shaped such that the plane separating the first portion and the second portion includes the opening central axis but does not include the rotational axis. In some implementations, the first portion and the second portion are separated along a plane that extends perpendicular to the rotational axis and includes the opening central axis.

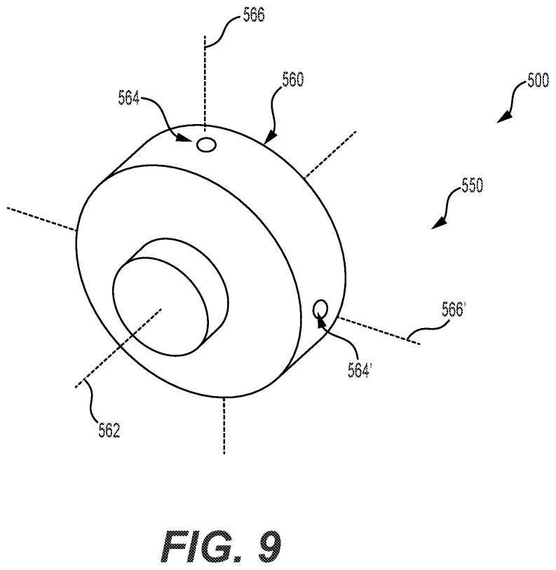

FIG. 9 shows another implementation of a device 500 including a needle holder portion 550 defining a first needle opening 564 and a second needle opening 564′. The first needle opening 564 has a first opening central axis 566, and the second needle opening 564′ has a second opening central axis 566′. Both the first opening central axis 566 and the second opening central axis 566′ extend perpendicularly to the rotational axis 562 of the body 560. The needle holder portion 550 is configured such that, when the coupling body 510 is coupled to an ultrasound probe 190, the first opening central axis 566 and the second opening central axis 566′ are disposed within the scanning plane 196 in all positions in the range from a first position to a second position of each respective needle opening 564.

The first needle opening 564 has a first diameter and the second needle opening 564′ has a second diameter that is different than the first diameter. In this implementation, the needle holder portion 550 does not need to be removed and replaced with a different needle holder portion defining a different needle opening with a different diameter. Instead, the needle holder portion 550 can be rotated until the other needle opening 564, 564′ with the desired diameter can be used.

Although the needle holder portion 550 shown in FIG. 9 defines two needle openings 564, 564′, in other implementations, the needle holder portion defines three or more needle openings.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

What is claimed is:

1. An ultrasound needle guidance device, the device comprising:
a coupling body configured to fixedly couple to an ultrasound probe, wherein the ultrasound probe is able to produce an ultrasonic beam along a scanning plane; and
at least one needle holder portion, wherein the needle holder portion has a rotational axis and defines a needle opening having an opening central axis extending perpendicularly to the rotational axis, wherein the needle holder portion is rotationally coupled to the coupling body such that the needle holder portion is rotatable about the rotational axis relative to the coupling body from a first position to a second position, wherein the needle holder portion is configured such that, when the coupling body is coupled to the ultrasound probe, the opening central axis is disposed within the scanning plane in all positions in a range from the first position to the second position.

2. The device of claim 1, wherein the needle holder portion comprises a round cross-sectional shape in a plane perpendicular to the rotational axis.

3. The device of claim 1, wherein the needle holder portion comprises one or more protrusions extending radially relative to the rotational axis, wherein the needle opening extends through the protrusion.

4. The device of claim 1, wherein the ultrasound probe has a scanning surface plane through which the ultrasonic beam passes, wherein the scanning plane is perpendicular to the scanning surface plane, wherein the opening central axis is disposed at a 0-degree angle relative to the scanning surface plane in the first position.

5. The device of claim 4, wherein the opening central axis is disposed at a 90-degree angle relative to the scanning surface plane in the second position.

6. The device of claim 1, wherein the needle opening has a diameter from about 0.203 mm to about 1.473 mm.

7. The device of claim 1, wherein the coupling body further comprises a collar, the collar defining a probe opening configured to receive an ultrasound probe, wherein the probe opening has an inner surface and an outer surface.

8. The device of claim 7, wherein the coupling body further comprises a holder support, the holder support comprising two arms extending from the outer surface of the collar.

9. The device of claim 8, wherein the needle holder portion comprises portions configured to be disposed within the arms such that the needle holder portion is removably couplable to the coupling body.

10. The device of claim 8, wherein each arm is arcuate.

11. The device of claim 10, wherein the arms have an inner radius of curvature and the portions of the needle holder portion have an inner radius of curvature, and wherein the inner radius of curvature of the arms is substantially the same as the inner radius of curvature of the portions of the needle holder portion.

12. The device of claim 1, wherein the needle opening is a first needle opening having a first opening central axis, the needle holder portion further defining a second needle opening having a second opening central axis extending perpendicularly to the rotational axis, wherein the needle holder portion is configured such that, when the coupling body is coupled to the ultrasound probe, the second opening central axis is disposed within the scanning plane in all positions in the range from the first position to the second position.

13. The device of claim 12, wherein the first needle opening has a first diameter and the second needle opening has a second diameter that is different than the first diameter.

14. The device of claim 1, wherein the needle holder portion is removably couplable to the coupling body.

15. The device of claim 14, wherein the needle holder portion comprises a first portion and a second portion that is separable from the first portion, wherein at least one surface of the first portion and at least one surface of the second portion abut each other when the needle holder portion is coupled to the coupling body, wherein the at least one surface of the first portion and the at least one surface of the second portion define the needle opening.

16. The device of claim 1, wherein the coupling body includes an inner surface configured to face the ultrasound probe when the device is coupled to the ultrasound probe, wherein the inner surface of the coupling body includes a protrusion configured to engage at least one feature of the ultrasound probe.

17. The device of claim 1, wherein the coupling body includes an inner surface configured to face the ultrasound probe when the device is coupled to the ultrasound probe, wherein the inner surface of the coupling body defines an opening configured to engage at least one feature of the ultrasound probe.

18. The device of claim 1, wherein the coupling body further includes a latch configured to engage at least one feature of the ultrasound probe.

19. The device of claim 18, wherein the latch is resiliently biased toward an engagement position in which the latch is configured to engage the at least one feature of the ultrasound probe and urgable toward a disengagement position in which the latch is configured to disengage the at least one feature of the ultrasound probe.

20. The device of claim 1, wherein the coupling body comprises a resilient material.
